(19) United States
(12) Patent Application Publication
(10) Pub. No.: US 2014/0081062 A1
(43) Pub. Date: Mar. 20, 2014

(54) Process for the Preparation of Butadiene with Removal of Oxygen from C4-Hydrocarbon Streams

(71) Applicant: BASF SE, Ludwigshafen (DE)

(72) Inventors: Alireza Rezai, Mannheim (DE); Gauthier Luc Maurice Averlant, Frankfurt (DE); Martin Dieterle, Ludwigshafen (DE); Godwind Tafara Peter Mabande, Novi, MI (US)

(73) Assignee: BASF SE, Ludwigshafen (DE)

(21) Appl. No.: 14/032,390

(22) Filed: Sep. 20, 2013

Related U.S. Application Data
(60) Provisional application No. 61/703,279, filed on Sep. 20, 2012.

(51) Int. Cl. C07C 5/48

(52) U.S. Cl. CPC C07C 5/48
USPC 585/326

(57) ABSTRACT

A process for preparing butadiene from n-butane by two-step dehydrogenation and removal of the residual oxygen comprised in the gas stream by means of a catalytic combustion stage which is carried out in the presence of a catalyst which comprises a monolith which comprises a catalytically inert material having a low BET surface area and a catalyst layer which has been applied to the monolith and comprises an oxidic support material, at least one noble metal selected from the group consisting of the noble metals of group VIII of the Periodic Table of the Elements, optionally tin and/or rhenium, and optionally further metals, where the thickness of the catalyst layer is from 5 to 500 μm, is described.

The invention relates to a process for the preparation of butadiene with removal of oxygen from C₄₋hydrocarbon streams comprising free oxygen.

In the preparation of butadiene from butane C₄-hydrocarbon streams comprising free oxygen can be obtained and the free oxygen should be or has to be removed from these since it can lead to the formation of peroxides which are difficult to handle from a safety point of view.

WO 2006/075025 describes a process for preparing butadiene from n-butane by nonoxidative catalytic dehydrogenation of n-butane, subsequent oxidative dehydrogenation and workup of the product mixture. After the oxidative dehydrogenation, the oxygen remaining in the product gas stream can be removed, for example by reacting it catalytically with hydrogen. A corresponding C₄ product gas stream can comprise from 20 to 80% by volume of butadiene, from 20 to 80% by volume of n-butane, from 0.5 to 50% by volume of 2-butene and from 0 to 20% by volume of 1-butene and also small amounts of oxygen. The residual oxygen can cause problems since it can act as initiator for polymerization reactions in downstream process steps. This risk is particularly great when butadiene is separated off by distillation and can there lead to deposition of polymers (formation of “popcorn”) in the extractive distillation column. A removal of oxygen is therefore carried out immediately after the oxidative dehydrogenation, generally by means of a catalytic combustion step in which oxygen is reacted with the hydrogen comprised in the gas stream in the presence of a catalyst. Here, a reduction in the oxygen content down to small traces is achieved. α-Aluminum oxide comprising from 0.01 to 0.1% by weight of platinum and from 0.01 to 0.1% by weight of tin is described as suitable catalyst. As an alternative, catalysts comprising copper in reduced form are also reported.

WO 2010/130610 describes a process for preparing propylene oxide by reaction of propene with hydrogen peroxide and isolation of the propylene oxide to give a gas mixture comprising propene and oxygen. Hydrogen is added to this gas mixture and the oxygen comprised is at least partly reacted by reaction with the hydrogen in the presence of a copper-comprising catalyst. Here, the catalyst comprises from 30 to 80% by weight of copper, calculated as CuO.

WO 2010/113565 relates to a monolith catalyst and its use. A monolith catalyst comprising Pt, Sn, K, Cs and La on an SiO₂/ZrO₂ mixed oxide as support is used for the dehydrogenation of alkanes to alkenes, in particular of propane to propene or n-butane to butenes, or for the catalytic combustion of hydrogen by means of oxygen.

Apart from “popcorn” formation, the oxygen content in hydrocarbon-comprising gas mixtures, in particular gas mixtures comprising butadiene and oxygen, can contribute to deactivation of catalysts, to soot deposits, peroxide formation, to a deterioration in the adsorption properties of solvents in the work-up process.

Particularly in the preparation of butadiene from n-butane, selective oxygen removal is a basic prerequisite for carrying out the process economically since every loss of the target product butadiene is associated with increased costs. The specification to be met is a residual oxygen concentration after the oxygen removal step of less than 100 ppm.

It is an object of the present invention to provide an improved process for the catalytic removal of oxygen from C₄-hydrocarbon mixtures. The catalyst should make it possible to catalyze the selective reaction of free oxygen with free hydrogen when the hydrocarbon stream comprises free hydrogen, without appreciable amounts of C₄-hydrocarbons, in particular butadiene, also being reacted.

The object is achieved according to the invention by a process for preparing butadiene from n-butane, which comprises the steps
A) provision of a feed gas stream a comprising n-butane;
B) introduction of the feed gas stream a comprising n-butane into at least one first dehydrogenation zone and nonoxidative catalytic dehydrogenation of n-butane to give a gas stream b comprising n-butane, 1-butene, 2-butenes, butadiene, hydrogen, possibly water vapor, possibly carbon oxides and possibly inert gases;
G) introduction of a stream f which comprises butane, butenes, butadiene and has been obtained from the gas stream b and of an oxygen-comprising gas into at least one second dehydrogenation zone and oxidative dehydrogenation of 1-butene and 2-butenes to give a gas stream g comprising n-butane, unreacted 1-butene and 2-butenes, butadiene, water vapor, possibly carbon oxides, possibly hydrogen and possibly inert gases and
H) removal of the residual oxygen comprised in the gas stream g by means of a catalytic combustion stage in which the oxygen is reacted with part or all of the hydrogen d2 which has previously been separated off and/or additionally introduced hydrogen to give an oxygen-depleted stream h,
   wherein step H) is carried out in the presence of a catalyst which comprises a monolith which comprises a catalytically inert material having a low BET surface area and a catalyst layer which has been applied to the monolith and comprises an oxidic support material, at least one noble metal selected from the group consisting of the noble metals of group VIII of the Periodic Table of the Elements, optionally tin and/or rhenium and optionally further metals, where the thickness of the catalyst layer is from 5 to 500 μm.

The catalyst used in step H) will firstly be described in more detail.

The catalyst preferably comprises platinum and tin.

The catalyst layer preferably comprises a metal of the third transition group of the Periodic Table of the Elements including the lanthanides, in particular lanthanum.

The catalyst layer preferably comprises at least one alkali metal or alkaline earth metal, particularly preferably potassium and/or cesium.

The oxidic support material is preferably selected from among oxides of the metals of the second, third and fourth main groups and the third and fourth transition groups. The support material is particularly preferably selected from among oxides of magnesium, calcium, aluminum, silicon, titanium and zirconium or mixtures thereof, in particular from among silicon dioxide (SiO₂) and/or zirconium dioxide (ZrO₂).

The monolith is preferably made up of cordierite.

The catalyst used in step H) is known per se and is described, for example, in WO 2010/133565. As regards the precise make-up of the catalyst and its production, reference can be made to this document.

The fixed-bed catalyst used has a significantly reduced noble metal content and improved performance. The penetration depth of the catalyst is restricted to from 5 to 500 μm, preferably from 5 to 250 μm, in particular from 25 to 250 μm, especially from 50 to 250 μm. The penetration depth of the catalyst is limited by the catalyst layer applied to the monolith.

The catalyst layer on the monolith comprises at least one ceramic oxide as catalyst support and at least one noble metal selected from among the elements of transition group VIII of the Periodic Table of the Elements, especially palladium, platinum or rhodium, optionally rhenium and/or tin. A catalyst support is made up of one or more ceramic oxides of elements of the second, third and fourth main groups and the third and fourth transition groups (group IV B) of the Periodic Table of the Elements and the lanthanides, especially MgO, CaO, Al₂O₃, SiO₂, ZrO₂, TiO₂, La₂O₃ and Ce₂O₃. In a particularly preferred embodiment, the catalyst support comprises SiO₂ and ZrO₂; it is, in particular, a mixed oxide of SiO₂ and ZrO₂.

In addition to the noble metals of transition group VIII, it is possible to use further elements in the catalytically active layer, for example rhenium and/or tin. Furthermore, doping with compounds of the third main group or transition group (III A or III B) or basic compounds such as alkali metals, alkaline earth metals or rare earths or compounds thereof which can be converted into the corresponding oxides at temperatures above 400° C. can be effected. Simultaneous doping with a plurality of the elements mentioned or compounds thereof is possible. Suitable examples are potassium and lanthanum compounds. In addition, the catalyst can be mixed with sulfur, tellurium, arsenic, antimony or selenium or compounds thereof, which in many cases lead to an increase in the selectivity.

The catalyst layer comprises at least one noble metal from group VIII of the Periodic Table of the Elements (Ru, Rh, Pd, Os, Ir, Pt). The preferred noble metal is platinum. The catalyst layer can optionally comprise tin and/or rhenium. It preferably comprises tin.

In a preferred embodiment, the catalyst layer comprises platinum and tin.

In addition, the catalyst layer can be doped with further metals, for example with metals of the third transition group (group III B) of the Periodic Table of the Elements, including the lanthanides (Sc, Y, La, Ce, Pr, Nd, Sm, Eu, Gd, Tb, Dy, Ho, Er, Tm, Yb, Lu). Preference is given to using cerium and lanthanum, in particular lanthanum.

In addition, the catalyst layer can comprise metals selected from among the metals of main groups I and II of the Periodic Table of the Elements. The catalyst layer preferably comprises potassium and/or cesium.

Especial preference is given to a catalyst layer comprising platinum, tin, lanthanum, potassium and cesium.

The catalyst layer is applied to the monolith by washcoating. It is also possible firstly to apply the catalyst support layer composed of the oxidic support material to the monolith by washcoating and subsequently impregnate this layer with one or more different solutions of the metals or metal compounds.

Suitable monolith structures can be metallic or ceramic. They are preferably made up of individual blocks having small (0.5 to 4 mm in diameter) parallel channels. Particular preference is given to using cordierite as material for the monolithic structures. Suitable monoliths frequently have a low BET surface area, in the case of cordierite, for example, 0.7 m²/g. For the purposes of the invention, a low BET surface area is a BET surface area of less than 10 m²/g.

For a more precise description of the cordierite monoliths, reference may be made to WO 2010/133565, pages 4 to 6.

The monolithic structure is coated with a catalyst support layer (washcoat comprising at least one ceramic oxide) or a catalyst layer which comprises the catalytically active metals on the ceramic oxide support layer. With regard to the washcoating process, reference can likewise be made to WO 2010/133565, in particular pages 6 to 12.

The proportion of elements of transition group VIII and optionally rhenium or tin in the catalyst can be in the range from 0.005 to 5% by weight, preferably from 0.1 to 2% by weight, in particular from 0.05 to 1.5% by weight. When rhenium or tin are additionally used, the ratio of these to the noble metal can be in the range from 0.1:1 to 20:1, preferably from 1:1 to 10:1.

The catalyst material usually has a BET surface area of up to 500 m²/g, preferably from 2 to 300 m²/g, in particular from 5 to 300 m²/g. The pore volume is usually in the range from 0.1 to 1 ml/g, preferably from 0.15 to 0.6 ml/g, in particular from 0.2 to 0.4 ml/g. The average pore diameter of the mesopores, determined by mercury penetration analysis, is generally from 8 to 60 nm, preferably from 10 to 50 nm.

The proportion of pores having a pore size of greater than 20 nm is usually in the range from 0 to 90%.

As regards the production of the catalytically active layer including the catalyst support, reference can again be made to WO 2010/133565, in particular pages 12 to 18. The amount of noble metal in the catalysts is preferably from 0.005 to 1% by weight, in particular from 0.05 to 0.5% by weight.

The catalyst used according to the invention in step H) has the advantage that, in particular, it catalyzes the reaction of hydrogen with oxygen without an appreciable reaction of C₄-hydrocarbon, in particular butadiene, with the free oxygen occurring.

In a preferred process according to the invention, the following steps C) to F) are carried out between steps B) and G), with the stream f being introduced in step G):
C) compression in at least one first compression stage and cooling of the gas stream b, to give at least one condensate stream c1 comprising water and a stream c2 comprising butenes and butadiene, n-butane, hydrogen, water vapor, possibly carbon oxides and possibly inert gases;
D) absorption of the butenes and of the stream c2 comprising butadiene, n-butane, hydrogen, water vapor, possibly inert gases and possibly carbon oxides by means of a selective solvent, e.g. a mixture comprising from 80 to 97% by weight of N-methylpyrrolidone and from 3 to 20% by weight of water, to give a stream d1 comprising selective solvent such as N-methylpyrrolidone, water and butenes, butadiene, butane and possibly carbon dioxide and a stream d2 comprising hydrogen and possibly inert gases and butane;
E) extractive distillation of the selective solvent, e.g. stream d1 comprising N-methylpyrrolidone, water and butenes, butadiene, butane and possibly carbon oxides by means of a selective solvent, e.g. a stream e1 comprising from 80 to 97% by weight of N-methylpyrrolidone and from 3 to 20% by weight of water, with the selective solvent, e.g. stream d1 comprising N-methylpyrrolidone, water and butenes, butadiene, butane and possibly carbon oxides being separated into a stream e2 comprising selective solvents such as N-methylpyrrolidone, water and butane, butenes, butadiene and a stream e3 comprising essentially butane and possibly carbon oxides;
F) distillation of the selective solvent, e.g. stream e2 comprising N-methylpyrrolidone, water, butane and butenes, butadiene to give a stream e1 comprising essentially selective solvents such as N-methylpyrrolidone and water and a stream f comprising butane, butenes, butadiene.

Preference is given to recirculating all or part of the stream d2 to the first dehydrogenation zone B).

An additional feed stream can be introduced in step G).

Preference is given to recirculating all or part of the stream e1 to the absorption zone D) and the extractive distillation zone E).

Preference is given to recirculating all or part of the stream e3 to step A).

Carbon oxides are carbon dioxide, carbon monoxide or mixtures thereof.

The following steps I) to L) are preferably carried out after H):
I) compression in at least a first compression stage and cooling of the oxygen-depleted stream h or gas stream g to give at least one condensate stream i1 comprising water and a gas stream i2 comprising n-butane, 1-butene, 2-butenes, butadiene, hydrogen, water vapor, possibly carbon oxides and possibly inert gases;
J) separation of the incondensable and low-boiling gas constituents comprising hydrogen, oxygen, carbon oxides, the low-boiling hydrocarbons methane, ethane, ethene, propane, propene and inert gases as gas stream j2 from the gas stream i2 to give a C₄ product gas stream j1 which consists essentially of C₄-hydrocarbons;
K) separation of the gas stream j1 by extractive distillation by means of a selective solvent, preferably a mixture comprising from 80 to 97% by weight of N-methylpyrrolidone and from 3 to 20% by weight of water, into a stream k1 consisting essentially of butadiene and selective solvent, preferably N-methylpyrrolidone, or comprising these and a stream k2 comprising n-butane, butenes, water vapor and possibly inert gases;
L) distillation of the selective solvent, preferably stream k1 comprising N-methylpyrrolidone, water and butadiene to give a stream l1 comprising essentially selective solvents, preferably N-methylpyrrolidone and water, and a stream l2 comprising butadiene.

The following step M) is preferably carried out after step L):
M) pure distillation of the stream 12 comprising butadiene in one or two columns, in which a stream m2 comprising butadiene is obtained and a gas stream m1 comprising impurities which are more volatile than butadiene and/or a bottom stream m3 comprising impurities which are less volatile than butadiene is/are separated off.

Preference is given to recirculating all or part of the gas stream j2 to the second dehydrogenation zone G).

Preference is given to recirculating all or part of the stream k2 to the feed gas stream in step A), the absorption step D), the extraction step E) and/or in part to the second dehydrogenation zone G).

The separation in step J) is preferably carried out in two stages by absorption with subsequent desorption.

Preference is given to recirculating all or part of the stream l1 to the step K).

The nonoxidative catalytic dehydrogenation of n-butane is preferably carried out autothermally with introduction of an oxygen-comprising gas. The oxygen-comprising gas can be, for example, air, oxygen-enriched air or technical-grade oxygen.

The feed stream a comprising n-butane can have been obtained from liquefied petroleum gas (LPG).

The process of the invention preferably allows optimal utilization of the butane introduced and optimized operation of a second dehydrogenation step by means of the indicated work-up after the first dehydrogenation step.

The present separation task can be carried out using selective solvents whose affinity to C₄-hydrocarbons having single bonds increases in the direction of C₄-hydrocarbons having double bonds and further to conjugated double bonds and triple bonds, preferably dipolar, particularly preferably dipolar aprotic solvents. For apparatus reasons, substances which cause little corrosion or are noncorrosive are preferred.

Suitable selective solvents for the process of the invention are, for example, butyrolactone, nitriles such as acetonitrile, propionitrile, methoxypropionitrile, ketones such as acetone, furfurol, N-alkyl-substituted lower aliphatic acid amides such as dimethylformamide, diethylformamide, dimethylacetamide, diethylacetamide, N-formylmorpholine, N-alkyl-substituted cyclic acid amides (lactams) such as N-alkylpyrrolidones, in particular N-methylpyrrolidone. In general, alkyl-substituted lower aliphatic acid amides of N-alkyl-substituted cyclic acid amides are used. Dimethylformamide, acetonitrile, furfurol and in particular N-methylpyrrolidone are particularly advantageous.

However, it is also possible to use mixtures of these solvents with one another, for example N-methylpyrrolidone with acetonitrile, mixtures of these solvents with cosolvents such as water and/or tert-butyl ethers, for example methyl tert-butyl ether, ethyl tert-butyl ether, propyl tert-butyl ether, n-butyl or isobutyl tert-butyl ether.

N-Methylpyrrolidone, hereinafter referred to as NMP for short, is particularly useful, preferably in aqueous solution, advantageously with from 0 to 20% by weight of water.

According to the invention, preference is given to using a mixture of from 80 to 97% by weight of N-methylpyrrolidone and from 3 to 20% by weight of water, preferably a mixture of from 90 to 93% by weight of N-methylpyrrolidone and from 7 to 10% by weight of water and in particular a mixture of from 91 to 92% by weight of N-methylpyrrolidone and from 8 to 9% by weight of water, for example a mixture of 91.7% by weight of N-methylpyrrolidone and 8.3% by weight of water, both as solvent for the absorption in step D) and as extractant for the extraction in step E) and in step K).

Compared to the production of butadiene by steam cracking, the process displays a high selectivity. No undesirable coproducts are obtained. The complicated separation of butadiene from the product gas mixture from the cracking process is dispensed with.

The process of the invention displays particularly effective utilization of the raw materials. Thus, losses of the raw material n-butane are minimized by the preferred recirculation of unreacted n-butane from process step E) to the first dehydrogenation step. The n-butane is not (completely) passed through the process steps F)-J), as a result of which these apparatuses can be made smaller.

The preferred separation of butene and butane after the first dehydrogenation step and preferred recirculation of the butane results in a higher conversion of butane into butene than when a butane/butene mixture from the extractive distillation is recirculated after the second dehydrogenation step. Unreacted butene is preferably recirculated to the second dehydrogenation step. The partial isolation of a butene-comprising product stream having a butene content which can be set by means of the extractive distillation is possible here.

It is also possible to feed a butene-comprising C₄ stream in addition to stream f into the oxidative dehydrogenation step G). This stream can originate from all butene-comprising sources. Conceivable streams are, for example, FCC product streams and butene-comprising streams produced by dimerization of ethylene.

The individual steps can be carried out as described in DE-A-10 2004 059 356, DE-A-10 2004 054 766 and DE-A-10 2004 061 514.

Preferred ways of carrying out the process are described below:

In a first process section A, a feed gas stream a comprising n-butane is provided. n-Butane-rich gas mixtures such as liquefied petroleum gas (LPG) are usually employed as raw material. LPG comprises essentially saturated C₂₋C₅-hydrocarbons. In addition, it also comprises methane and traces of C₅⁺-hydrocarbons. The composition of LPG can vary greatly. The LPG used advantageously comprises at least 10% by weight of n-butane.

As an alternative, it is possible to use an upgraded C₄ stream from crackers or refineries.

In a variant of the process of the invention, the provision of the dehydrogenation feed gas stream comprising n-butane comprises the steps
A1) provision of a liquefied petroleum gas (LPG) stream,
A2) separation of propane and possibly methane, ethane and C₅⁺-hydrocarbons (mainly pentanes, also hexanes, heptanes, benzene, toluene) from the LPG stream to give a stream comprising butanes (n-butane and isobutane),
A3) separation of isobutane from the stream comprising butanes to give the feed gas stream comprising n-butane and optionally isomerization of the isobutane which has been separated off to give an n-butane/isobutane mixture and recirculation of the n-butane/isobutane mixture to the removal of isobutene.

The removal of propane and possibly methane, ethane and C₅⁺-hydrocarbons is, for example, carried out in one or more conventional rectification columns. For example, low boilers (methane, ethane, propane) can be separated off at the top of a first column and low boilers (C₅⁺-hydrocarbons) can be separated off at the bottom of a second column. This gives a stream comprising butanes (n-butane and isobutane) from which isobutane is separated off in, for example, a conventional rectification column. The remaining, n-butane-comprising stream is used as feed gas stream for the subsequent butane dehydrogenation.

The isobutane stream which has been separated off can be subjected to isomerization. For this purpose, the isobutane-comprising stream is introduced into an isomerization reactor. The isomerization of isobutane to n-butane can be carried out as described in GB-A 2018815. This gives an n-butane/isobutane mixture which is introduced into the n-butane/isobutane separation column.

The isobutane stream which has been separated off can also be passed to a further use, for example for preparing methacrylic acid, polyisobutene or methyl tert-butyl ether.

The feed gas stream a comprising n-butane generally comprises at least 60% by weight of n-butane, preferably at least 90% by weight of n-butane. In addition, it can comprise C₁-C₄-hydrocarbons as secondary constituents.

In a process section B, the feed gas stream comprising n-butane is introduced into a dehydrogenation zone and subjected to a nonoxidative catalytic dehydrogenation. Here, n-butane is partially dehydrogenated to 1-butene and 2-butenes over a dehydrogenation-active catalyst in a dehydrogenation reactor, with butadiene (1,3-butadiene) also being formed. In addition, hydrogen and small amounts of methane, ethane, ethene, propane and propene are obtained. Depending on the way in which the dehydrogenation is carried out, the product gas mixture from the nonoxidative catalytic dehydrogenation of n-butane can additionally comprise carbon oxides (CO, CO₂), water and inert gases such as nitrogen. In addition, unreacted n-butane is present in the product gas mixture.

A feature of the nonoxidative mode of operation compared to an oxidative mode of operation is that no free hydrogen is formed in the oxidative dehydrogenation.

The nonoxidative catalytic dehydrogenation of n-butane can in principle be carried out in all reactor types and modes of operation known from the prior art. A description of dehydrogenation processes which are suitable for the purposes of the invention may also be found in “Catalytica® Studies Division, Oxidative Dehydrogenation and Alternative Dehydrogenation Processes” (Study Number 4192 OD, 1993, 430 Ferguson Drive, Mountain View, Calif., 94043-5272, USA).

The nonoxidative catalytic dehydrogenation of butane can be carried out with or without an oxygen-comprising gas as cofeed. It is preferably carried out as an autothermal nonoxidative dehydrogenation with introduction of oxygen as cofeed. In the autothermal mode of operation, the heat required is generated directly in the reactor system by combustion of hydrogen and/or hydrocarbons in the presence of oxygen. A hydrogen-comprising cofeed can preferably be additionally mixed in. Oxygen is additionally mixed into the reaction gas mixture for the dehydrogenation of n-butane in at least one reaction zone and the hydrogen and/or hydrocarbon comprised in the reaction gas mixture is at least partially burnt, as a result of which at least part of the dehydrogenation heat required in the at least one reaction zone is generated directly in the reaction gas mixture. Preference is given to operation using pure oxygen. Oxygen can preferably be introduced as oxygen/steam mixture or as air/steam mixture. The use of an oxygen/steam mixture introduces only small amounts of inert gases (nitrogen) into the overall process.

In general, the amount of oxygen-comprising gas added to the reaction gas mixture is selected so that the quantity of heat required for the dehydrogenation of butane is generated by the combustion of hydrogen present in the reaction gas mixture and optionally hydrocarbons present in the reaction gas mixture and/or of carbon present in the form of carbonaceous deposits. In general, the total amount of oxygen introduced is, based on the total amount of butane, from 0.001 to 0.5 mol/mol, preferably from 0.005 to 0.2 mol/mol, particularly preferably from 0.05 to 0.2 mol/mol.

The hydrogen burnt for the generation of heat is the hydrogen formed in the catalytic dehydrogenation of butane and optionally hydrogen additionally added as hydrogen-comprising gas to the reaction gas mixture. The amount of hydrogen present should preferably be such that the molar ratio in the reaction gas mixture immediately after the introduction of oxygen is from 1 to 10 mol/mol, preferably from 2 to 5 mol/mol. In the case of multistage reactors, this applies to each intermediate introduction of oxygen-comprising and optionally hydrogen-comprising gas.

The combustion of hydrogen proceeds catalytically. The dehydrogenation catalyst used generally also catalyzes the combustion of hydrocarbons and of hydrogen by means of oxygen, so that no specific oxidation catalyst is necessary in principle. Suitable catalysts are described, for example, in DE-A 10 2004 061 514.

Suitable reactors are all reactors known to those skilled in the art for the use of heterogeneous catalysts for gas-solid catalysis.

In an embodiment of the process of the invention, intermediate introduction of oxygen-comprising gas and of hydrogen-comprising gas is effected before each tray of a tray reactor. In a further embodiment of the process of the invention, the introduction of oxygen-comprising gas and of hydrogen-comprising gas is effected before each tray apart from the first tray. In an embodiment, a layer of a specific oxidation catalyst followed by a layer of the dehydrogenation catalyst is present after each point of introduction. In a further embodiment, no specific oxidation catalyst is present. Suitable catalysts are described, for example, in DE-A 10 2004 061 514, see also WO 2009/124974 and WO 2009/124945 and particularly preferably WO 2010/133565. The dehydrogenation temperature is generally from 400 to 1100° C., and the pressure in the outlet from the reactor is generally from 0.2 to 5 bar, preferably from 1 to 3 bar. The space velocity (GHSV) is generally from 500 to 2000 h⁻¹, in the case of high-load operation also up to 100 000 h⁻¹, preferably from 4000 to 16 000 h⁻¹.

Other reactors such as monolith reactors are also suitable.

Preference is given to a reactor (1) in the form of a horizontal cylinder or prism for carrying out an autothermal gas-phase dehydrogenation of a butane-comprising gas stream (2) by means of an oxygen-comprising gas stream (3) to give a reaction gas mixture over a heterogeneous catalyst configured as monolith (4), wherein
the interior space of the reactor (1) is divided by a cylindrical or prismatic gastight housing G arranged in the longitudinal direction of the reactor (1) into
an inner region A which has one or more catalytically active zones (5) in each of which a packing made up of monoliths (4) stacked on top of one another, next to one another and behind one another is provided and in which a mixing zone (6) having fixed internals is provided upstream of each catalytically active zone (5) and
an outer region B arranged coaxially with the inner region A, where
a heat exchanger (12) is provided at one end of the reactor attached to the housing G, with one or more feed lines (7) for the butane-comprising gas stream (2) to be dehydrogenated,
with one or more independently regulable feed lines (9), where each feed line (9) conveys the oxygen-comprising gas stream (3) from one or more distribution chambers (10) to each of the mixing zones (6) and
with a discharge line (11) for the reaction gas mixture from the autothermal gas-phase dehydrogenation, where
the outer region B is supplied with a gas which is inert under the reaction conditions of the autothermal gas-phase dehydrogenation and
the butane-comprising gas stream (2) to be dehydrogenated is introduced via a feed line (7) into the heat exchanger (12), is heated in countercurrent by indirect heat exchange with the reaction gas mixture in the heat exchanger (12) and is conveyed further to the end of the reactor opposite the heat exchanger (12), is deflected there, introduced via a flow equalizer (8) into the inner region A and is mixed in the mixing zones (6) with the oxygen-comprising gas stream (3), whereupon the autothermal gas-phase dehydrogenation takes place in the inner region A of the reactor (1).

The gas which is inert under the reaction conditions of the autothermal gas-phase dehydrogenation is preferably water vapor.

The gas which is inert under the reaction conditions of the autothermal gas-phase dehydrogenation is preferably conveyed as purge gas stream at a mass flow from ⅕ to 1/100, preferably from 1/10 to 1/50, based on the mass flow of the butane-comprising gas stream (2) under a low gauge pressure of from 2 to 50 mbar, preferably from 25 to 30 mbar, based on the pressure in the inner region A through the outer region B, preferably by introducing the purge gas stream at one end of the reactor via one or more feed lines (20) into the outer region B of the reactor and conveying it further at the opposite end of the reactor into the inner region A of the reactor, in particular via one or more connecting line(s) (21) which is/are advantageously arranged at an angle different from 90° to the feed line (7) for the butane-comprising gas stream (2) to be dehydrogenated.

The butane-comprising gas stream (2) to be dehydrogenated is preferably introduced at one or more points into the heat exchanger (12), preferably as a main stream having a relatively high mass flow and one or more secondary streams having a mass flow lower than that of the main stream.

In addition to the heat exchanger (12), one or more supplementary heating facilities for the butane-comprising gas stream (2) to be dehydrogenated are preferably provided.

The introduction of hydrogen via a line (23) into the feed line (7) for the butane-comprising gas stream (2) to be dehydrogenated, ideally close to the inlet into the mixing zones (6) which are arranged upstream of each catalytically active zone (5), is preferably provided as additional heating facility for the butane-comprising gas stream (2), with an electric heating element (22), which is preferably installed in a detachable manner, as a plug-in system, inside the outer region B of the reactor (1) or as a muffle burner (22) in the feed line (7) for the butane-comprising gas stream (2) to be dehydrogenated after exit of the latter from the heat exchanger (12) being able to be provided as supplementary heating facility.

Two or more catalytically active zones (5) each having a packing made up of monoliths (4) stacked on top of one another, next to one another and behind one another are preferably provided in the inner region A.

Two or more of the reactors (1) can be used, with at least one reactor (1) being utilized for the autothermal gas-phase dehydrogenation and at the same time at least one further reactor (1) being regenerated.

The regeneration is preferably carried out in a temperature range from 550 to 700° C.

The regeneration is preferably carried out using an oxygen-comprising gas stream comprising from 0.1 to 1.5% by weight of oxygen, based on the total weight of the oxygen-comprising gas stream.

In this reactor,
the interior space of the reactor is divided by a cylindrical or prismatic gastight housing G arranged in a detachable manner in the longitudinal direction of the reactor into
an inner region A which has one or more catalytically active zones in each of which a packing made up of monoliths stacked on top of one another, next to one another and behind one another are provided and in which a mixing zone having fixed internals is provided upstream of each catalytically active zone and
an outer region B arranged coaxially with the inner region A, where
a heat exchanger is provided at one end of the reactor attached to the housing G,
with one or more feed lines for the butane-comprising gas stream to be dehydrogenated,
with one or more independently regulable feed lines, where each feed line conveys the oxygen-comprising gas stream from one or more distribution chambers to each of the mixing zones and
with a discharge line for the reaction gas mixture from the autothermal gas-phase dehydrogenation,
   where the outer region B is supplied with a gas which is inert under the reaction conditions of the autothermal gas-phase dehydrogenation and the butane-comprising gas stream to be dehydrogenated is introduced via a feed line into the heat exchanger, is heated in countercurrent by indirect heat exchange with the reaction gas mixture and is conveyed further to the end of the reactor opposite the heat exchanger, is deflected there, introduced via a flow equalizer into the inner region A and is mixed in the mixing zones with the oxygen-comprising gas stream, whereupon the autothermal gas-phase dehydrogenation takes place in the inner region A of the reactor.

The autothermal gas-phase dehydrogenation takes place over a heterogeneous catalyst which is present in the form of monoliths.

According to the invention, the individual monoliths are stacked next to one another, above one another, and behind one another in the number required to fill out a catalytically active zone and form a packing.

A mixing zone having fixed internals which are not catalytically active is provided upstream of each packing. The mixing of the butane-comprising gas stream with the oxygen-comprising stream occurs in the mixing zone, with the mixing of the oxygen-comprising gas stream with the butane-comprising feed stream occurring in the first mixing zone into which flow occurs in the flow direction and intermediate introduction of an oxygen-comprising gas stream into the butane-comprising reaction mixture still to be hydrogenated occurring in each of the subsequent mixing zones into which flow occurs.

The butane-comprising gas stream to be dehydrogenated can preferably be introduced at two or more points into the heat exchanger, in particular as a main stream having a relatively high mass flow and one or more secondary streams having a mass flow lower than that of the main stream.

The dehydrogenation of butane is generally preferably carried out in the presence of water vapor. The added water vapor serves as heat transfer medium and aids gasification of organic deposits on the catalysts, which counters the formation of carbonaceous deposits on the catalysts and increases the operating life of the catalysts. The organic deposits are converted into carbon monoxide, carbon dioxide and possibly water.

The nonoxidative catalytic dehydrogenation of n-butane gives a gas mixture which comprises not only butadiene, 1-butene, 2-butenes and unreacted n-butane but generally also secondary constituents. Usual secondary constituents are hydrogen, water vapor, CO₂ and also low boilers (methane, ethane, ethene, propane and propene). The composition of the gas mixture leaving the first dehydrogenation zone can vary greatly as a function of the way in which the dehydrogenation is carried out. Thus, when the preferred autothermal dehydrogenation with introduction of oxygen and additional hydrogen is carried out, the product gas mixture has a comparatively high content of water vapor and carbon oxides. In modes of operation without introduction of oxygen, the product gas mixture of the nonoxidative dehydrogenation has a comparatively high content of hydrogen.

The product gas stream from the nonoxidative autothermal dehydrogenation of n-butane preferably comprises from 0.1 to 15% by volume of butadiene, from 1 to 15% by volume of 1-butene, from 1 to 25% by volume of 2-butene (cis/trans-2-butene), from 20 to 70% by volume of n-butane, from 1 to 70% by volume of water vapor, from 0 to 10% by volume of low-boiling hydrocarbons (methane, ethane, ethene, propane and propene), from 0.1 to 40% by volume of hydrogen, from 0 to 10% by volume of inert gas (nitrogen) and from 0 to 5% by volume of carbon oxides, where the total amount of the constituents is 100% by volume.

The product gas stream b leaving the first dehydrogenation zone can, after compression in process step C), be divided in process step D) into two substreams, with only one of the two substreams being subjected to the further process sections E) to M) and the second substream being recirculated to the first dehydrogenation zone. A corresponding mode of operation is described in DE-A 102 11 275. However, it is also possible for the entire product gas stream b from the nonoxidative catalytic dehydrogenation of n-butane to be subjected to the further process sections E) to M).

In process step C), the gas stream b is preferably firstly cooled. Cooling of the compressed gas is carried out using heat exchangers which can, for example, be configured as shell-tube, spiral or plate heat exchangers. The heat removed is preferably utilized for heat integration in the process. In a preferred embodiment of process step C), water is subsequently separated off from the product stream. The water is preferably separated off in a quench.

The gas stream c is subsequently compressed in at least one first compression stage and subsequently cooled, with at least one condensate stream c1 comprising water being condensed out and a gas stream c2 comprising n-butane, 1-butene, 2-butenes, butadiene, hydrogen, water vapor, small amounts of methane, ethane, ethene, propane and propene and possibly carbon oxides and possibly inert gases remaining.

The compression can be carried out in one or more stages. Overall, the gas stream is compressed from a pressure in the range from 1.0 to 4.0 bar to a pressure in the range from 3.5 to 20 bar. Each compression stage is followed by a cooling stage in which the gas stream is cooled to a temperature in the range from 15 to 60° C. The condensate stream c1 can thus also comprise a plurality of streams in the case of multistage compression.

The gas stream c2 generally consists essentially of C₄-hydrocarbons (essentially n-butane, 1-butene and 2-butenes), hydrogen, carbon dioxide and water vapor. In addition, the stream c2 can comprise low boilers, butadiene and inert gases (nitrogen) as further secondary components. The condensate stream c1 generally comprises at least 80% by weight, preferably at least 90% by weight, water and additionally comprises small amounts of low boilers, C₄-hydrocarbons, oxygenates and carbon oxides.

Suitable compressors are, for example, turbocompressors, rotary piston compressors and reciprocating piston compressors. The compressors can, for example be driven by an electric motor, an expander or a gas or steam turbine. Typical compression ratios (exit pressure: inflow pressure) per compressor stage are, depending on the construction type, in the range from 1.5 to 3.0.

The cooling of the compressed gas is carried out by means of heat exchangers which can, for example, be configured as shell-and-tube, spiral or plate heat exchangers. Cooling water or heat transfer oils are generally used as coolants in the heat exchangers. In addition, preference is given to using air cooling using blowers.

The absorption in step (D) can be carried out in any suitable absorption column known to those skilled in the art. This absorption is preferably carried out in countercurrent. For this purpose, the stream comprising butenes, butadiene, butane, hydrogen, inert gas (nitrogen) and possibly carbon oxides is fed into the lower region of the absorption column. In the upper region of the absorption column, the stream comprising N-methylpyrrolidone and water is introduced.

A stream d2 which is richer in hydrogen and/or richer in inert gas (nitrogen) and may still comprise residues of O₄-hydrocarbons and possibly carbon oxygenates is taken off at the top of the absorption column. The stream can further comprise inerts (for example nitrogen) and low boilers (ethane, ethene, propane, propene, methane). The stream comprising N-methylpyrrolidone and water cools the stream comprising butenes and/or butadiene, butane, hydrogen and/or inert gas (nitrogen) and possibly carbon oxides which is fed in and at the same time preferably absorbs the C₄ components and some of the carbon oxides. Small amounts of H₂, inerts (N₂) and low boilers may also be absorbed. This stream is taken off at the bottom of the absorption column.

The use of a mixture of N-methylpyrrolidone and water as solvent for the absorption and as extractant in the extractive distillation has the advantage that the boiling point is lower than the boiling point when using pure N-methylpyrrolidone. A further advantage is that the selectivity can be increased by increasing the proportion of water in the mixture of water and N-methylpyrrolidone used as solvent. However, this leads, as expected, to a reduction in the capacity. A further advantage is the selectivity of N-methylpyrrolidone over carbon oxides, in particular carbon dioxide. This makes it possible, in addition to separating off the hydrocarbons, to separate the carbon oxides, in particular carbon dioxide, from the hydrogen.

The absorption in step D) is generally carried out at a temperature at the bottom in the range from 30 to 160° C., a temperature at the top in the range from 5 to 60° C. and a pressure in the range from 2 to 20 bar. The absorption is preferably carried out at a temperature at the bottom in the range from 30 to 100° C., at a temperature at the top in the range from 25 to 50° C. and a pressure in the range from 8 to 15 bar.

The absorption column is preferably a column having random packing elements or ordered packing. However, any other column, for example a tray column, is also conceivable. A column suitable for the absorption preferably has from 2 to 40 theoretical plates, preferably from 5 to 25 theoretical plates.

The temperature of the stream comprising N-methylpyrrolidone and water, e.g. e1 and/or k2, which is fed to the absorption column is preferably from 10 to 70° C., more preferably from 20 to 40° C. The temperature of the stream comprising butenes, butadiene, butane, hydrogen and/or inert gas (nitrogen) and possibly carbon oxides is preferably in the range from 0 to 400° C., in particular in the range from 40 to 200° C.

The ratio of N-methylpyrrolidone used to the stream comprising butenes, butadiene, butane, hydrogen and/or inert gas and possibly carbon oxides is preferably in the range from 2 to 30, more preferably in the range from 4 to 30 and in particular in the range from 4 to 15, in each case based on the masses of the streams used.

The stream d1 comprising N-methylpyrrolidone, water, butenes, butadiene, butane and carbon oxides which is obtained in the absorption generally comprises from 20 to 90 mol % of N-methylpyrrolidone, from 0 to 50 mol % of water, from 0 to 20 mol % of butadiene, from 0 to 20 mol % of 1-butene, from 0 to 20 mol % of 2-butenes, from 0 to 50 mol % of butane and from 0 to 20 mol % of carbon oxides.

The stream d1 comprising N-methylpyrrolidone, water, butenes, butadiene, butane and carbon oxides which is obtained in the absorption is then fed to an extractive distillation in step (E).

The extractive distillation can, for example, be carried out as described in Erdöl and Kohle Erdgas-Petrochemie volume 34 (8), pages 343 to 346 or Ullmanns Enzyklopädie der technischen Chemie, volume 9, 4^{th} edition 1975, pages 1 to 18.

In the extractive distillation, the stream d1 comprising butenes, butadiene, butane, methylpyrrolidone, water and carbon oxides is brought into contact with a stream comprising N-methylpyrrolidone and water in an extractive distillation zone. The extractive distillation zone is generally in the form of a column comprising trays, random packing elements or ordered packing as internals. The extractive distillation zone generally has from 10 to 70 theoretical plates so as to achieve a sufficiently good separation performance. The extraction column preferably has a backwashing zone at the top of the column. This backwashing zone serves to recover the N-methylpyrrolidone comprised in the gas phase by means of liquid hydrocarbon runback, for which purpose the overhead fraction is condensed beforehand. Typical temperatures at the top of the column are in the range from 30 to 60° C.

The overhead product stream e3 from the extractive distillation column comprises butane and carbon oxides and is taken off in gaseous form. The overhead product stream can comprise not only butane and carbon oxides but also butenes, hydrogen and/or inert gas and other low boilers. In a preferred embodiment, the overhead product stream e3 is condensed in order to separate off carbon oxides such as CO₂ and any hydrogen and/or inert gas and low boilers present from butane. The liquid butane stream can, for example, be recirculated to the dehydrogenation zone in process step B).

A stream e2 comprising N-methylpyrrolidone, water, butenes, butane and butadiene is obtained at the bottom of the extractive distillation column. Overhead removal of part of the butane serves to concentrate the butenes in the stream e2. The degree of concentration can be set via the parameters of the column.

The stream e2 comprising N-methylpyrrolidone, water, butane, butenes and butadiene and is obtained at the bottom of the extractive distillation column is fed to a distillation column F) from which a stream f consisting essentially of butenes, butane and butadiene is obtained at the top. A stream e1) comprising N-methylpyrrolidone and water is obtained at the bottom of the distillation column, with the composition of the stream comprising N-methylpyrrolidone and water corresponding to the composition introduced into the absorption and the extraction. The stream comprising N-methylpyrrolidone and water is preferably divided and conveyed back into the absorption in process step D) and the extractive distillation in process step E). The ratio of the mixture of water and N-methylpyrrolidone which is fed to the absorption to the mixture of water and N-methylpyrrolidone and O₄ which is fed to the extractive distillation is preferably in the range from 0.2 to 20, in particular in the range from 0.3 to 15.

The stream f separated off at the top can be partly or completely taken from the plant and used as product stream. Here, the butene content can be set via the way in which the extractive distillation is operated. A high butene concentration reduces the amount of butane which has to be conveyed through process step G) and the subsequent process steps. At the same time, this increases the yield of the BDH stage.

The extractive distillation is preferably operated at a temperature at the bottom in the range from 90 to 250° C., in particular at a temperature in the range from 90 to 210° C., a temperature at the top in the range from 10 to 100° C., in particular in the range from 20 to 70° C., and a pressure in the range from 1 to 15 bar, in particular in the range from 3 to 8 bar. The extractive distillation column preferably has from 5 to 70 theoretical plates.

The distillation in process step (F) is preferably carried out at a temperature at the bottom in the range from 100 to 300° C., in particular in the range from 150 to 200° C., and a temperature at the top in the range from 0 to 70° C., in particular in the range from 10 to 50° C. The pressure in the distillation column is preferably in the range from 1 to 10 bar. The distillation column preferably has from 2 to 30, in particular from 5 to 20, theoretical plates.

Apart from the stream f obtained from process step F), further n-butene-comprising streams as are obtained, for example, in refineries from FCC units or by dimerization of ethylene can also be fed to the ODH stage in process step G). According to the invention, the addition of any n-butene-comprising stream is conceivable.

Essentially 1-butene and 2-butenes are dehydrogenated to 1,3-butadiene in the oxidative (catalytic) dehydrogenation in process step G), with 1-butene generally reacting virtually completely.

The oxidative dehydrogenation can in principle be carried out in all types of reactor known from the prior art and using known modes of operation, for example in a fluidized bed, in a tray oven, in a fixed-bed tube or shell-and-tube reactor or in a plate heat exchanger reactor. To carry out the oxidative dehydrogenation, a gas mixture having a molar oxygen:n-butenes ratio of at least 0.5 is preferably required. Preference is given to working at an oxygen:n-butenes ratio of from 0.55 to 50, preferably from 0.55 to 10, in particular from 0.55 to 3. To set this value, the product gas mixture coming from the nonoxidative catalytic dehydrogenation is generally mixed with pure oxygen or an oxygen-comprising gas, either directly or after a work-up in which butenes are concentrated and hydrogen is separated off. In an embodiment of the process, the oxygen-comprising gas is air. The oxygen-comprising gas mixture obtained is then fed to the oxydehydrogenations. As a preferred alternative to air, additional nitrogen or lean air can be used in a proportion of less than 23% by volume as oxygen-comprising gas. In a preferred embodiment, the offgas from process step J), viz. stream j2, is mixed with the stream f and optionally additional steam and fed to process step G). The amount of nitrogen which may be required for diluting the stream f can thereby be reduced or made superfluous.

Catalysts which are particularly suitable for the oxydehydrogenation are generally based on an Mo—Bi—O-comprising multimetal oxide system which generally additionally comprises iron. In general, the catalyst system comprises further additional components from groups 1 to 15 of the Periodic Table, for example potassium, magnesium, zirconium, chromium, nickel, cobalt, cadmium, tin, lead, germanium, lanthanum, manganese, tungsten, phosphorus, cerium, aluminum or silicon.

Suitable catalysts and their production are described, for example, in U.S. Pat. No. 4,423,281, U.S. Pat. No. 4,336,409, DE-A-2600128 and DE-A-2440329 and also WO 2009/124974 and WO 2009/124945.

The catalyst for the oxydehydrogenation is generally used as shaped bodies having an average size of greater than 2 mm. Owing to the appreciable pressure drop while carrying out the process, smaller shaped bodies are generally unsuitable. Suitable shaped bodies which may be mentioned by way of example are pellets, cylinders, hollow cylinders, rings, spheres, extrudates, wagon wheels or extrudates. Particular shapes such as “trilobes” and “tristars” (see EP-A-0 593 646) or shaped bodies having at least one notch on the outside (see U.S. Pat. No. 5,168,090) are likewise possible.

In general, the catalyst used can be employed as all-active catalyst. In this case, the entire shaped catalyst body consists of the active composition, including possible auxiliaries such as graphite or pore formers, and also further components. Furthermore, it is possible to apply the active compositions of the catalysts, including possible auxiliaries such as graphite or pore formers, and also further components to a support, for example an inorganic, oxidic shaped body. Such catalysts are generally referred to as coated catalysts.

The oxydehydrogenation is generally carried out at a temperature of from 220 to 490° C. and preferably from 250 to 450° C. A reactor inlet pressure which is sufficient to overcome the flow resistances present in the plant and the subsequent work-up is selected. This reactor inlet pressure is generally from 0.005 to 1 MPa gauge, preferably from 0.01 to 0.5 MPa gauge. The gas pressure applied in the inlet region of the reactor naturally drops substantially over the total catalyst bed.

The product gas stream g leaving the oxidative dehydrogenation comprises butadiene and n-butane which has not been separated off in process step E) together with hydrogen, carbon oxides and water vapor. As secondary constituents, it can further comprise oxygen, inert gas such as nitrogen, methane, ethane, ethene, propane and propene and also oxygen-comprising hydrocarbons, known as oxygenates.

In general, the product gas stream g leaving the oxidative dehydrogenation comprises from 2 to 40% by volume of butadiene, from 5 to 80% by volume of n-butane, from 0 to 15% by volume of 2-butenes, from 0 to 5% by volume of 1-butene, from 5 to 70% by volume of water vapor, from 0 to 10% by volume of low-boiling hydrocarbons (methane, ethane, ethene, propane and propene), from 0.1 to 15% by volume of hydrogen, from 0 to 70% by volume of inert gas, from 0 to 10% by volume of carbon oxides, from 0 to 10% by volume of oxygen and from 0 to 10% by volume of oxygenates, where the total amount of the constituents is 100% by volume. Oxygenates can be, for example, furan, acetic acid, methacrolein, maleic anhydride, maleic acid, phthalic anhydride, propionic acid, acetaldehyde, acrolein, formaldehyde, formic acid, benzaldehyde, benzoic acid and butyraldehyde. In addition, acetylene, propyne and 1,2-butadiene can be comprised in traces.

If the product gas stream g comprises more than only slight traces of oxygen, a process step H) is generally carried out to remove residual oxygen from the product gas stream g. The residual oxygen can interfere insofar as it can cause butadiene peroxide formation in downstream process steps and can act as initiator for polymerization reactions.

Unstabilized 1,3-butadiene can form dangerous butadiene peroxides in the presence of oxygen. The peroxides are viscous liquids. Their density is higher than that of butadiene. Since they are also only sparingly soluble in liquid 1,3-butadiene, they settle out at the bottom of storage vessels. Despite their relatively low chemical reactivity, the peroxides are very unstable compounds which can decompose spontaneously at temperatures in the range from 85 to 110° C. A particular hazard is the high shock sensitivity of the peroxides, which explode with the brisance of an explosive.

The risk of polymer formation is present in particular in the isolation by distillation of butadiene (steps L and M) and can there lead to deposits of polymers (formation of “popcorn”) in the columns.

The removal of oxygen H) is preferably carried out directly after the oxidative dehydrogenation G). In general, a catalytic combustion stage in which oxygen is reacted in the presence of a catalyst with hydrogen introduced into this stage is carried out for this purpose. This hydrogen can be taken off as part of the stream d2 from process step D). In this way, a reduction in the oxygen content down to small traces is achieved.

The hydrocarbon stream comprising free oxygen can comprise an amount of free hydrogen which is sufficient for the reaction with the free oxygen. Missing amounts or the total amount of free hydrogen required can be added to the hydrocarbon stream. In this way of carrying out the reaction, the free oxygen can be reacted with the free hydrogen so that no appreciable proportion of the hydrocarbon is reacted with the oxygen.

In an alternative embodiment, the hydrocarbon stream comprising free oxygen does not comprise any free hydrogen and no free hydrogen is added to it either. In this case, the free oxygen can be reacted with the hydrocarbon comprised in the hydrocarbon stream comprising free oxygen or with added methanol, natural gas and/or synthesis gas as reducing agent.

The process can be carried out isothermally or adiabatically. The advantage of the reaction of the hydrogen is the formation of water as reaction product. The water formed can easily be separated off by condensation.

Since butadiene is a reactive molecule, low reaction temperatures are advantageous in the removal of oxygen. This makes it possible to achieve high selectivities and prevent uncontrolled homogeneous reactions.

In addition, a low reaction pressure can be advantageous since this makes it possible to avoid a separate compression step after the oxidative dehydrogenation. A low reaction pressure allows less costly reactor manufacture and is advantageous for safety reasons.

Step H) of the process of the invention is therefore preferably carried out at a pressure of from 0.5 to 3.0 bar (absolute), particularly preferably from 1.0 to 2.0 bar (absolute).

The reaction is preferably carried out at a temperature in the range from 100 to 650° C., particularly preferably from 250 to 550° C.

The type of reactor is not subject to any restrictions according to the invention. For example, the reaction can be carried out in a fluidized bed, in a tray oven, in a fixed-bed tube or shell-and-tube reactor or in a plate heat exchanger reactor. Cascading of fluidized-bed reactors is also conceivable.

The heat evolved in the reaction can be removed via the reactor walls. In addition, the formation of hot spots can be avoided by structuring of a fixed bed of the catalyst with inert materials.

If hydrogen is used in an above stoichiometric amount in step H) of the process of the invention, the reaction with hydrogen can serve to achieve a sufficiently high temperature for the necessary reaction between hydrocarbons and oxygen. Formation of carbonaceous deposits can be largely avoided in this way.

If no hydrogen or a substoichiometric amount of hydrogen is used, the oxygen reacts predominantly with the most reactive molecule, for example butadiene. This results in formation of carbon oxides and water. Since the reaction of oxygen with the hydrocarbons proceeds more slowly than with hydrogen at low temperature, the hydrogen is completely consumed first.

In a further embodiment of the invention, this catalytic reaction is carried out together with the oxidative dehydrogenation in process step G in a reactor having 2 catalysts and optionally intermediate introduction of the combustion gas downstream of the dehydrogenation bed.

In process step I), the gas stream h is preferably firstly cooled. Cooling of the compressed gas is effected by means of heat exchangers which can be configured, for example, as shell-and-tube, spiral or plate heat exchangers. The heat removed here is preferably utilized for heat integration in the process. In a preferred embodiment of process step I), water is subsequently separated off from the product stream. The water is preferably separated in a quench. The quench additionally serves to separate off oxygen-comprising by-products.

The gas stream h is subsequently compressed in at least one first compression stage and then cooled, resulting in at least one condensate stream i1 comprising water being condensed out and a gas stream i2 comprising n-butane, 1-butene, 2-butenes, butadiene, possibly hydrogen, water vapor and small amounts of methane, ethane, ethene, propane and propene, carbon oxides and inert gases remaining.

The compression can be carried out in one or more stages. Overall, the gas stream is compressed from a pressure in the range from 1.0 to 4.0 bar to a pressure in the range from 3.5 to 20 bar. Each compression stage is followed by a cooling stage in which the gas stream is cooled to a temperature in the range from 15 to 60° C. The condensate stream i1 can thus also comprise a plurality of streams in the case of multistage compression.

The condensate stream i1 generally comprises at least 80% by weight, preferably at least 90% by weight, of water and additionally comprises small amounts of low boilers, C₄-hydrocarbons, oxygenates and carbon oxides.

Suitable compressors are, for example turbocompressors, rotary piston compressors and reciprocating piston compressors. The compressors can, for example, be driven by an electric motor, an expander or a gas or steam turbine. Typical compression ratios (exit pressure:inflow pressure) per compression stage are, depending on the construction type, in the range from 1.5 to 3.0.

Cooling of the compressed gas is carried out by means of heat exchangers which can, for example, be configured as shell-and-tube, spiral or plate heat exchangers. Cooling water or heat transfer oils are used as coolants in the heat exchangers. Furthermore, preference is given to using air cooling using blowers.

The stream i2 comprising butadiene, butenes, butane, hydrogen, inert gas and possibly carbon oxides and also low-boiling hydrocarbons (methane, ethane, ethene, propane, propene) is fed as starting stream to step J) of the workup.

In a preferred embodiment of the process of the invention, the incondensable or low-boiling gas constituents such as hydrogen, oxygen, carbon oxides, the low-boiling hydrocarbons (methane, ethane, ethene, propane, propene) and inert gas such as nitrogen are separated off by means of a high-boiling absorption medium in an absorption/desorption cycle, giving a O₄ product gas stream j1 which consists essentially of the C₄-hydrocarbons. In general, the O₄ product gas stream j1 comprises at least 80% by volume, preferably at least 90% by volume, particularly preferably at least 95% by volume, of the C₄-hydrocarbons. The stream j1 consists essentially of n-butane, butenes such as 2-butenes and butadiene.

For this purpose, the product gas stream i2 is, after prior removal of water, brought into contact with an inert absorption medium in an absorption stage and the C₄-hydrocarbons are absorbed in the inert absorption medium, giving absorption medium loaded with C₄-hydrocarbons and an offgas j2 comprising the above gas constituents. In a desorption stage, the C₄-hydrocarbons are liberated again from the absorption medium.

The absorption stage in step J) can be carried out in any suitable absorption column known to those skilled in the art. Absorption can be effected by simply passing the product gas stream i2 through the absorption medium. However, it can also be carried out in columns or in rotary absorbers. These can be operated in cocurrent, countercurrent or cross-current. The absorption is preferably carried out in countercurrent. Suitable absorption columns are, for example, tray columns having bubble cap trays, centrifugal trays and/or sieve trays, columns having structured packing, e.g. sheet metal packing having a specific surface area of from 100 to 1000 m²/m³, e.g. Mellapak® 250 Y, and columns packed with random packing elements. However, trickle and spray towers, graphite block absorbers, surface absorbers such as thick film and thin film absorbers and also rotary columns, plate scrubbers, cross-spray scrubbers and rotary scrubbers are also suitable.

In an embodiment of the invention, the stream comprising butadiene, butene, butane, hydrogen and/or nitrogen and possibly carbon dioxide is fed into the lower region of an absorption column. The stream comprising solvent and optionally water is introduced in the upper region of the adsorption column.

Absorption media according to the invention are octanes, nonanes, decanes, undecanes, dodecanes, tridecanes, tetradecanes, pentadecanes, hexadecanes, heptadecanes and octadecanes or fractions which are obtained from refinery streams and comprise the abovementioned linear alkanes as main components.

Further suitable absorption media are comparatively nonpolar organic solvents, for example aliphatic C₈-C₁₈-alkenes, or aromatic hydrocarbons such as middle oil fractions from paraffin distillation, or ethers having bulky groups or mixtures of these solvents, with a polar solvent such as 1,2-dimethyl phthalate being able to be added to these. Further suitable absorption media are esters of benzoic acid and phthalic acid with straight-chain C₁-C₈-alkanols, e.g. n-butyl benzoate, methyl benzoate, ethyl benzoate, dimethyl phthalate, diethyl phthalate and also heat transfer oils such as biphenyl and diphenyl ether, chloro derivatives thereof and triarylalkenes. One suitable absorption medium is a mixture of biphenyl and diphenyl ether, preferably with the azeotropic composition, for example the commercially available Diphyl®. This solvent mixture frequently comprises dimethyl phthalate in an amount of from 0.1 to 25% by weight.

In a preferred embodiment, an alkane mixture such as tetradecane (industrial C14-C17 fraction) is used as solvent for the absorption in step J).

An offgas stream j2 which comprises essentially inert gas, carbon oxides, possibly butane, butenes such as 2-butenes and butadiene, possibly hydrogen and low-boiling hydrocarbons (methane, ethane, ethene, propane, propene) and water vapor is taken off at the top of the absorption column. This stream j2 is fed to the process step G). The feed stream to the ODH reactor can in this way be set to the desired C₄ content.

The solvent stream loaded with C₄-hydrocarbons is introduced into a desorption column. According to the invention, all column internals known to those skilled in the art are suitable for this purpose. In a process variant, the desorption step is carried out by depressurization and/or heating of the loaded solvent. A preferred process variant is the addition of stripping steam and/or the introduction of fresh steam in the bottom of the desorber. The C₄-depleted solvent can be fed as a mixture together with the condensed steam (water) to a phase separation, so that the water is separated off from the solvent. All apparatuses known to those skilled in the art are suitable for this purpose. An additional possibility is utilization of the water separated off from the solvent for generating the stripping steam. Preference is given to using from 70 to 100% by weight of solvent and from 0 to 30% by weight of water, particularly preferably from 80 to 100% by weight of solvent and from 0 to 20% by weight of water, in particular from 85 to 95% by weight of solvent and from 5 to 15% by weight of water.

The separation J) is generally not quite complete, so that, depending on the type of separation, small amounts or even only traces of the further gas constituents, in particular the low-boiling hydrocarbons, can still be present in the C₄ product gas stream. The volume flow reduction brought about by the separation J) also relieves the load on the subsequent process steps.

The C₄ product gas stream j1 consisting essentially of n-butane, butenes such as 2-butenes and butadiene generally comprises from 20 to 80% by volume of butadiene, from 20 to 80% by volume of n-butane, from 0 to 10% by volume of 1-butene and from 0 to 50% by volume of 2-butenes, where the total amount is 100% by volume.

In a process section K), the C₄ product gas stream j1 is separated into a recycle stream k2 consisting essentially of n-butane and butenes such as 2-butenes and a stream k1 consisting essentially of butadiene by extractive distillation. The stream k2 is preferably added to the feed gas stream in step A) and/or (partly) recirculated to the absorption stage in process step D), the extraction step E) and/or process step G) (ODH reactor).

The extractive distillation K) can be carried out, for example, as described in Erdöl and Kohle Erdgas-Petrochemie, volume 34 (8), pages 343 to 346, or Ullmanns Enzyklopädie der technischen Chemie, volume 9, 4^{th} edition 1975, pages 1 to 18.

The extractive distillation is preferably carried out at a temperature at the bottom in the range from 100 to 250° C., in particular at a temperature in the range from 110 to 210° C., a temperature at the top in the range from 10 to 100° C., in particular in the range from 20 to 70° C., and a pressure in the range from 1 to 15 bar, in particular in the range from 3 to 8 bar. The extractive distillation column preferably has from 5 to 70 theoretical plates.

In the extractive distillation, the stream comprising butenes, butadiene, butane, methylpyrrolidone and water is brought into contact with a stream as described above comprising N-methylpyrrolidone and water in an extractive distillation zone. The extractive distillation zone is generally in the form of one or more column(s) which comprise(s) trays, random packing elements or ordered packing as internals. The extractive distillation zone generally has from 10 to 70 theoretical plates so as to achieve a sufficiently good separation performance. The extraction column preferably has a backwashing zone at the top of the column. This backwashing zone serves to recover the N-methylpyrrolidone comprised in the gas phase by means of liquid hydrocarbon runback, for which purpose the overhead fraction is condensed beforehand. Typical temperatures at the top of the column are in the range from 30 to 60° C.

The overhead product stream k2 from the extractive distillation column comprises essentially butane and butenes and small amounts of butadiene and is taken off in gaseous or liquid form. In a preferred embodiment, the overhead product stream is condensed in order to separate off carbon oxides such as CO₂. The liquid butane/butene stream can be recirculated to the absorption column in process step A), D), E) and/or G). In this way, this stream goes together with the quenched, cooled, compressed product gas from the first dehydrogenation step which has been freed of condensate into the extractive distillation for the separation of butanes and butenes.

This separation of butane and butene then does not have to be carried out alongside the isolation of butadiene in the second extractive distillation.

At the bottom of the extractive distillation column, a stream k1 comprising N-methylpyrrolidone, water, butadiene and small amounts of butenes, butane is obtained and is fed to a distillation column L). In this, butadiene is obtained at the top or as side offtake stream. At the bottom of the distillation column, a stream l1 comprising N-methylpyrrolidone and water is obtained, with the composition of the stream comprising N-methylpyrrolidone and water corresponding to the composition as introduced into the extraction. The stream comprising N-methylpyrrolidone and water is preferably introduced into the extractive distillation in process step K).

The extractive distillation is preferably carried out at a temperature at the bottom in the range from 90 to 250° C., in particular at a temperature in the range from 90 to 210° C., a temperature at the top in the range from 10 to 100° C., in particular in the range from 20 to 70° C., and a pressure in the range from 1 to 15 bar, in particular in the range from 3 to 8 bar. The extractive distillation column preferably has from 5 to 70 theoretical plates.

The distillation in process step L) is preferably carried out at a temperature at the bottom in the range from 100 to 300° C., in particular in the range from 150 to 200° C., and a temperature at the top in the range from 0 to 70° C., in particular in the range from 10 to 50° C. The pressure in the distillation column is preferably in the range from 1 to 10 bar. The distillation column preferably has from 2 to 30, in particular from 5 to 20, theoretical plates.

A further distillation in process step M serves to purify the butadiene further and can be operated as described in the prior art.

The invention is illustrated by the following examples.

### EXAMPLE 1

### Production of the Pt/Sn/K/Cs/La Catalyst

The catalyst is a catalyst comprising Pt/Sn/K/Cs/La as active components on a ZrO₂/SiO₂ washcoat, with the washcoat being applied to a cordierite monolith. The ZrO₂ loading of the monolith is preferably in the range from 2 to 10 g/inch³.

A typical content, based on the total mass of the catalyst including the monolith, of the active components is 0.2% by weight of Pt, 0.4% by weight of Sn, 0.15% by weight of K, 0.2% by weight of Cs, 2.5% by weight of La, 30% by weight of Zr and 1.5% by weight of Si.

Platinum is the essential active metal here. Si and La stabilize the ZrO₂ support. K and Cs make the catalyst less acidic and thus reduce cracking reactions. The tin stabilizes, inter alia, the platinum dispersion.

In the active form of the catalyst, Cs, La, K and Si are fully oxidized and Pt is not oxidized. The tin is present in a small amount as alloy with Pt, in which both are metallic, and the major part of the tin is present in oxidic form.

ZrO₂, stabilized with SiO₂, is the porous support for the dopants K, Cs, La, Sn and Pt. ZrO₂ support extrudates which comprise 1% by weight of a polyethylene wax and from 10 to 15% by weight of starch as filler before the extrudates are calcined are used as starting material. The calcined extrudates are doped with La, K, Cs, Pt and Sn. The fillers are oxidized to carbon dioxide and water during the calcination, so that the finished catalyst does not comprise any fillers.

For the production of the catalyst, reference can be made to the examples of WO 2010/133565, in particular pages 22 to 25.

### EXAMPLE 2

### Removal of Oxygen in Step H

An oxygen removal reactor of a miniplant was used. The flow-through reactor had a length of 200 cm, an external diameter of 0.25 cm, a wall thickness of 0.02 cm and an internal diameter of 0.21 cm. It was made of steel.

The reactor was equipped with three external heating zones which were equipped with copper blocks for improved heat transfer from the heating elements to the reactor wall. To obtain an adiabatic system, the copper blocks were removed and replaced by insulation material in the second and third heating zones. The first heating zone was configured as a preheating zone in order to set the inlet gas temperature in the reactor. The second and third heating zones were configured so that heat losses were very largely prevented. The tube reactor was filled with catalyst only downstream of the end of the first heating zone. A pneumatically operated, multiple temperature sensor having four measurement points was used for determining the temperature profiles with a resolution of 2 cm in the catalyst bed. The catalyst bed was packed between an inert material (steatite), which served as guard bed. Both isothermal and adiabatic modes of operation were examined.

An alternative reactor on the laboratory scale had a length of 70 cm, an external diameter of 0.25 cm, a wall thickness of 0.02 cm and an internal diameter of 0.21 cm. It was made of steel.

Typical reaction conditions were a catalyst volume of 0.11, an amount of catalyst of from 0.01 to 0.1 kg, a GHSV of from 2000 to 10 000 standard I_{gas}I_{cat}⁻¹ h⁻¹, an inlet temperature of from 150 to 410° C. and an outlet pressure of from 1.5 to 2.5 bara.

A typical inlet gas stream comprised from 15 to 20% by volume of C_{d}-hydrocarbons (70% by volume of butadiene and 30% by volume of butane), from 10 to 20% by volume of water, from 5 to 10% by volume of hydrogen, from 50 to 60% by volume of nitrogen and from 3 to 5% by volume of oxygen.

In a mode of operation without hydrogen, the hydrogen was replaced by inert gas.

The objective of the process is to reduce the oxygen content to values of less than 100 ppm at the reactor outlet. In the case of processes without addition of hydrogen, the yields relate to CO₂ and traces of CO. In the process with use of hydrogen, the yields relate to CO₂ and CO and also dehydrogenation products of butadiene (butene isomers).

At a temperature of 410° C., a pressure of 0.5 bar/g and a GHSV of about 3000 h⁻¹, residual oxygen contents of 100 ppm were found for the catalyst from example 1 without addition of hydrogen and values of below 100 ppm were found when hydrogen was concomitantly used.

When, as an alternative, a catalyst comprising 28% by weight of copper on aluminum oxide was used, 108 ppm of residual oxygen were found without addition of hydrogen and 100 ppm of residual oxygen were found with addition of hydrogen.

The catalyst according to the invention met the requirements and at the same time displayed only very little formation of by-products.

1-14. (canceled)

15. A process for preparing butadiene from n-butane, which comprises the steps
A) provision of a feed gas stream a) comprising n-butane;
B) introduction of the feed gas stream a) comprising n-butane into at least one first dehydrogenation zone and nonoxidative catalytic dehydrogenation of n-butane to give a gas stream b) comprising n-butane, 1-butene, 2-butenes, butadiene, hydrogen, possibly water vapor, possibly carbon oxides and possibly inert gases;
G) introduction of a stream f) which comprises butane, butenes, butadiene and has been obtained from the gas stream b) and of an oxygen-comprising gas into at least one second dehydrogenation zone and oxidative dehydrogenation of 1-butene and 2-butenes to give a gas stream g) comprising n-butane, unreacted 1-butene and 2-butenes, butadiene, water vapor, possibly carbon oxides, possibly hydrogen and possibly inert gases and
H) removal of the residual oxygen comprised in the gas stream g) by means of a catalytic combustion stage in which the oxygen is reacted with part or all of the hydrogen d2) which has previously been separated off and/or additionally introduced hydrogen to give an oxygen-depleted stream h),
wherein step H) is carried out in the presence of a catalyst which comprises a monolith which comprises a catalytically inert material having a low BET surface area and a catalyst layer which has been applied to the monolith and comprises an oxidic support material, at least one noble metal selected from the group consisting of the noble metals of group VIII of the Periodic Table of the Elements, optionally tin and/or rhenium, and optionally further metals, where the thickness of the catalyst layer is from 5 to 500 μm.

16. The process according to claim 15 which comprises the following steps C) to F) between steps B) and G), with the stream f) being introduced in step G):
C) compression in at least one first compression stage and cooling of the gas stream b), to give at least one condensate stream c1) comprising water and a stream c2) comprising butenes and butadiene, n-butane, hydrogen, water vapor, possibly carbon oxides and possibly inert gases;
D) absorption of the butenes and of the stream c2) comprising butadiene, n-butane, hydrogen, water vapor, possibly inert gases and possibly carbon oxides by means of a first selective solvent to give a stream d1) comprising a second selective solvent and a stream d2) comprising hydrogen and possibly inert gases and butane;
E) extractive distillation of the second selective solvent by means of a third selective solvent with the second selective solvent being separated into a stream e2) comprising selected solvents selected from N-methylpyrrolidone, water and butane, butenes, and butadiene and a stream e3) comprising essentially butane and possibly carbon oxides;
F) distillation of the fourth selective solvent comprising selective solvents selected from N-methylpyrrolidone and water and a stream f) comprising butane, butenes, and butadiene.

17. The process according to claim 15, wherein all or part of the stream d2) is recirculated to the first dehydrogenation zone B) and/or all or part of the stream e1) is recirculated to the absorption step D) and the extractive distillation zone E) and/or all or part of the stream e3) is recirculated to step A).

18. The process according to claim 15, wherein the catalyst layer in step H) comprises platinum and tin.

19. The process according to claim 15, wherein the catalyst layer of the catalyst in step H) comprises a metal of the third transition group of the Periodic Table of the Elements including the lanthanides.

20. The process according to claim 15, wherein the catalyst layer of the catalyst in step H) comprises an alkali metal or alkaline earth metal.

21. The process according to claim 20, wherein the oxidic support material in the catalyst of step H) is selected from oxides of metals of the second, third and fourth main groups and the third and fourth transition groups; oxides of magnesium, calcium, aluminum, silicon, titanium, zirconium or mixtures thereof; ZrO2, SiO2, and mixtures of ZrO2 and SiO2.

22. The process according to claim 15, wherein the monolith in the catalyst of step H) comprises cordierite.

23. The process according to claim 15, wherein catalysts having an identical composition are used in steps B) and H).

24. The process according to claim 15, wherein the following steps I) to L) and optionally M) are carried out after step G) or H)
I) compression in at least a first compression stage and cooling of the oxygen-depleted stream h) or gas stream g) to give at least one condensate stream i1) comprising water and a gas stream i2) comprising n-butane, 1-butene, 2-butenes, butadiene, hydrogen, water vapor, possibly carbon oxides and possibly inert gases;
J) separation of the incondensable and low-boiling gas constituents comprising hydrogen, oxygen, carbon oxides, low-boiling hydrocarbons, methane, ethane, ethene, propane, propene and inert gases as gas stream j2) from the gas stream i2) to give a C₄ product gas stream j1) which consists essentially of C₄-hydrocarbons, with all or part of the gas stream j2) being able to be recirculated to the second dehydrogenation zone G) and the separation in step J) being able to be carried out in two stages by absorption with subsequent desorption;
K) separation of the gas stream j1) by extractive distillation by means of a selective solvent k3) into a stream k1) comprising butadiene and a selective solvent k4), and a stream k2) comprising n-butane, butenes, water vapor and possibly inert gases which can be recirculated in full or in part to the feed stream in step A), the absorption step D), the extraction step E) and/or in part to the second dehydrogenation zone G);
L) distillation of the selective solvent 13) to give a stream l1) comprising selective solvents selected from N-methylpyrrolidone and water, and a stream 12) comprising butadiene, with all or part of the stream l1) being able to be recirculated to the step K);
M) pure distillation of the stream l2) comprising butadiene in one or two columns, in which a stream m2) comprising butadiene is obtained and a gas stream m1) comprising impurities which are more volatile than butadiene and/or a bottom stream m3) comprising impurities which are less volatile than butadiene is/are separated off.

25. The process according to claim 15, wherein the nonoxidative catalytic dehydrogenation of n-butane is carried out autothermally with introduction of an oxygen-comprising gas.

26. The process according to claim 25, wherein air or oxygen-enriched air is introduced as oxygen-comprising gas or technical-grade oxygen is introduced as oxygen-comprising gas.

27. The process according to claim 15, wherein the feed gas stream a) comprising n-butane is obtained from liquefied petroleum gas (LPG).

28. The process according to claim 15, wherein an additional feed stream comprising butene is introduced in step G).

29. The process according to claim 15, wherein the first selective solvent is a mixture comprising from 80 to 97% by weight of N-methylpyrrolidone and from 3 to 20% by weight of water.

30. The process according to claim 15, wherein the second selective solvent is the stream d1).

31. The process according to claim 16, wherein the third selective solvent is a stream e1) comprising from 80 to 97% by weight of N-methylpyrrolidone and from 3 to 20% by weight of water.

32. The process according to claim 16, wherein the second selective solvent is the stream d1).

33. The process according to claim 16, wherein the fourth selective solvent is the stream e2) comprising N-methylpyrrolidone, water, butane and butenes, butadiene.

34. The process according to claim 19, wherein the catalyst layer of the catalyst in step H) comprises lanthanum.

35. The process according to claim 20, wherein the catalyst layer of the catalyst in step H) comprises potassium and/or cesium.

36. The process according to claim 24, wherein the selective solvent k3) is a mixture comprising from 80 to 97% by weight of N-methylpyrrolidone and from 3 to 20% by weight of water.

37. The process according to claim 24, wherein the selective solvent k4) is N-methylpyrrolidone.

38. The process according to claim 24, wherein the selective solvent l3) is the stream k1.
